(19) United States
(12) Patent Application Publication
(10) Pub. No.: US 2024/0166980 A1
(43) Pub. Date: May 23, 2024

(54) PHOTOBIOREACTOR FABRICATION SYSTEM

(71) Applicant: Korea University Research and Business Foundation, Seoul (KR)

(72) Inventors: Sang Jun SIM, Seoul (KR); Jeong Seop LEE, Seoul (KR); Ju Yeon LEE, Seoul (KR); Dong Hun KIM, Seoul (KR)

(73) Assignee: Korea University Research and Business Foundation, Seoul (KR)

(21) Appl. No.: 18/516,092

(22) Filed: Nov. 21, 2023

(30) Foreign Application Priority Data
Nov. 23, 2022 (KR) 10-2022-0158655

(51) Int. Cl. C12M 1/00; C08J 5/18

(52) U.S. Cl. CPC C12M 21/02; C08J 5/18

(57) ABSTRACT

A photobioreactor fabrication system is disclosed. According to a first embodiment, the photobioreactor fabrication system includes a first unwinder 10 continuously unwinding a first transparent film 1, a second unwinder 20 arranged parallel to the first unwinder 10 and continuously unwinding a second transparent film 2 such that the second film 2 faces the first film 1, a heat sealer 30 pressurizing and heat sealing the first 1 and second films 2 to a baffle 3 arranged between the first 1 and second films 2 facing each other, and a rewinder 40 simultaneously and continuously winding the first 1 and second films 2 having passed through the heat sealer 30.

### BACKGROUND OF THE INVENTION

### 1. Field of the Invention

The present invention relates to a photobioreactor fabrication system, and more specifically to a technology in which a roll-to-roll winder and a pressure heat sealer are used to continuously fabricate large-capacity photobioreactors.

### 2. Description of the Related Art

The phenomenon of global warming caused by large scale greenhouse gas emissions arising from the use of fossil fuels poses a threat to all living organisms on Earth, including human beings. Under these circumstances, carbon capture and storage (CCS) technologies for carbon dioxide reduction are actively being developed around the world. CCS technologies refer to methods in which a large amount of carbon dioxide released from a variety of carbon sources, including thermal power plants, is concentrated, captured, and injected into the underground or seabed to isolate it from the atmosphere. CCS technologies are effective in reducing a large amount of carbon dioxide in a short time, but the problems of unstable storage, difficult site selection, and high installation cost become substantial obstacles to the realization of CCS technologies in Korea. Thus, carbon capture and utilization (CCU) technologies for directly utilizing carbon dioxide in industrial applications or converting carbon dioxide into high value-added materials rather than sequestering carbon dioxide have recently received more attention than CCS technologies. In Korea, attempts have also been made to develop processes for converting carbon dioxide into high value-added materials. Particularly, biological processes for carbon dioxide conversion utilizing microalgae, photosynthetic microorganisms, have attracted attention as economical techniques that enable the production of various high value-added materials such as biofuels, bioplastics, and biopharmaceuticals, simultaneously with carbon dioxide reduction.

Microalgae, also called phytoplanktons, are aquatic unicellular organisms that photosynthesize. Microalgae are of increasing interest as highly potential biomass resources due to their ability to produce energy and industrial materials and reduce greenhouse gases. In the future, the utility value of microalgae is expected to rise in industrial fields, particularly energy, chemical, and environmental fields, for the following reasons.

First, in the energy field, microalgae have the highest oil productivity among all biodiesel producing crops. Soybean, canola, sunflower, oil palm, etc. have cultivation cycles of 4-8 months whereas microalgae can be cultivated on a daily basis due to their ability to proliferate several times in a day. The annual oil production from microalgae is at least 100 times that from soybean because microalgae have a higher fat content per unit weight. In addition, microalgae are bioresources free from the criticism that food resources are converted into energy and can produce biofuels with similar physical properties to petroleum diesel.

Second, in the chemical field, microalgae can produce numerous useful substances. Microalgae have been industrialized mainly in the field of food at present and will be industrialized in a wide range of applications, such as biochemicals and bioplastics, in the future. Health functional foods produced using Chlorella, Spirulina, and Chlamydomonas, which are microalgal species rich in protein, as well as Haematococcus that produces astaxanthin as a high value-added substance are currently commercially available to supplement various amino acids, antioxidants, and fatty acids.

Third, in the environmental field, microalgae have attracted the greatest attention due to their ability to reduce carbon dioxide, as described above, and their related studies have been continuously conducted all over the world. Microalgae can absorb carbon dioxide in an amount about twice the weight of biomass, which corresponds to a 10-50 times higher absorption efficiency than that of terrestrial plants. In addition, microalgae can be cultivated irrespective of specific soil and water quality. Related companies are extending the trials to utilize microalgae for carbon dioxide reduction and industrial wastewater purification.

The present inventors have developed a photobioreactor for effective microalgae cultivation that is composed of a transparent film (Korean Patent No. 10-1148194). However, the photobioreactor is disadvantageous in that only a very small amount of microalgae can be cultivated.

Since portions of the front/rear sides of the reactor are sealed to form internal corrugated compartments, scaling up of the reactor causes separation of the sealed portions, resulting in the disappearance of the internal compartments, and an increased amount of microalgal culture medium causes destruction of the internal structures, resulting in serious leakage at the bottom of the reactor.

Small-scale cultivation of microalgae has limitations in coping with increased carbon dioxide emissions released from large-scale industrial facilities (including coal-fired power plants and combined heat and power plants). To overcome these limitations, cultivation systems for microalgae are required to have a scale from a few tons to as many as hundreds of tons. Outdoor mass cultivation systems have been widely used around the world. In such a cultivation system, a reinforced concrete structure is manufactured to construct an open pond cultivation facility or a closed tempered glass or acrylic photobioreactor is utilized. However, the cultivation system has a problem in that the initial investment cost and management and maintenance cost increase considerably as the cultivation scale increases. The structural limitations of existing vinyl photobioreactors increase the risk of leakage and damage with increasing volume of culture medium in the reactors.

Thus, there is an urgent need for a solution to the problems of conventional photobioreactors.

### SUMMARY OF THE INVENTION

The present invention has been made in an effort to solve the problems of the prior art and one aspect of the present invention is to provide a photobioreactor fabrication system in which a hybrid of a roll-to-roll winder and a pressure heat sealer is used to continuously fabricate large-capacity photobioreactors for cultivating photosynthetic microorganisms.

A photobioreactor fabrication system according to an embodiment of the present invention includes a first unwinder continuously unwinding a first transparent film, a second unwinder arranged parallel to the first unwinder and continuously unwinding a second transparent film such that the second film faces the first film, a heat sealer pressurizing and heat sealing the first and second films to a baffle arranged between the first and second films facing each other, and a rewinder simultaneously and continuously winding the first and second films having passed through the heat sealer.

According to an exemplary embodiment of the present invention, the photobioreactor fabrication system may further include a first guide roller superimposing the unwound first and second films on each other and guiding the superimposed first and second films to the heat sealer.

According to an exemplary embodiment of the present invention, the baffle may include a first heat sealing member heat sealable to the first film, a second heat sealing member spaced apart from the first heat sealing member and heat sealable to the second film, and a partition member connecting the first and second heat sealing members and dividing a space between the first and second films.

According to an exemplary embodiment of the present invention, the baffle may be formed with a plurality of through holes penetrating the partition member.

According to an exemplary embodiment of the present invention, the photobioreactor fabrication system may further include a second guide roller arranged between the heat sealer and the rewinder and a third guide roller arranged to face the second guide roller in the vertical direction through the first and second films having passed through the heat sealer such that the first and second films are superimposed on each other and are guided to the rewinder.

According to an exemplary embodiment of the present invention, the photobioreactor fabrication system may further include a sensor unit measuring the moving distances of the first and second films facing each other after being unwound.

According to an exemplary embodiment of the present invention, the sensor unit may include a rotator rotating in contact with one of the first and second films moving while facing each other and an operation circuit measuring the rotation angle of the rotator to calculate the moving distances of the first and second films.

According to an exemplary embodiment of the present invention, the photobioreactor fabrication system may further include a baffle insertion unit inserting and arranging the baffle between the first and second films facing each other.

According to an exemplary embodiment of the present invention, the baffle insertion unit may include a first spacer roller guiding the first film to the heat sealer while rotating in contact with the lower surface of the first film unwound from the first unwinder, a second spacer roller spacing the second film from the first film and guiding the second film to the heat sealer while rotating in contact with the upper surface of the second film unwound from the second unwinder, and a baffle feeder feeding the baffle between the first and second films spaced apart from each other.

According to an exemplary embodiment of the present invention, the first and second films may be selected from the group consisting of low density polyethylene (LDPE) films, polyethylene terephthalate/cast polypropylene (PET+CPP) films, polyoxymethylene (POM) films, polycarbonate (PC) films, polyester sulfone (PES) films, polyethylene (PE) films, polyvinyl chloride (PVC) films, polyethylene terephthalate (PET) films, polypropylene (PP) films, polyphenylene oxide (PPO=PPE) films, and low density polyethylene/polyethylene terephthalate/Nylon 8 films.

The features and advantages of the present invention will become apparent from the following description with reference to the accompanying drawings.

Prior to the detailed description of the invention, it should be understood that the terms and words used in the specification and the claims are not to be construed as having common and dictionary meanings but are construed as having meanings and concepts corresponding to the technical spirit of the present invention in view of the principle that the inventor can define properly the concept of the terms and words in order to describe his/her invention with the best method.

The photobioreactor fabrication system of the present invention enables the fabrication of easy-to-scale-up photobioreactors in a continuous manner. In addition, the photobioreactor fabrication system of the present invention enables the fabrication of photobioreactors whose size can be adjusted, making it easy to control the scale of the reactors. Furthermore, the introduction of the roll-to-roll winder enables precise control over the movement of fabrics to reduce the reliance on the photobioreactor manufacturer's skill and can reduce the manufacturer's fatigue to improve the quality of the photobioreactors.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is a perspective view schematically illustrating a photobioreactor fabrication system according to a first embodiment of the present invention;

FIG. 2 is a side view schematically illustrating the photobioreactor fabrication system according to the first embodiment of the present invention;

FIG. 3 is a perspective view of the baffle illustrated in FIG. 2;

FIG. 4 illustrates a photobioreactor fabricated using the photobioreactor fabrication system according to the first embodiment of the present invention;

FIG. 5 is a side view schematically illustrating a photobioreactor fabrication system according to a second embodiment of the present invention;

FIG. 6 is a side view schematically illustrating a photobioreactor fabrication system according to a third embodiment of the present invention;

FIG. 7a is a schematic diagram of a tracer test to quantify the mass transfer rate of PBR and FIG. 7b shows the test results;

FIG. 8 compares CO₂ transfer efficiencies between a 70-L PBR (control) and a 150-L PBR (newly developed);

FIGS. 9a to 9d compare areal biomass productivities and PHB productivities between a 70-L PBR (control) and a 150-L PBR (newly developed). Specifically, FIG. 9a shows the biomass production per area of each PBR, FIG. 9b shows the PHB production per area and PHB content (%) of each PBR, FIG. 9c shows the maximum biomass productivity (gL⁻¹d⁻¹) of each PBR, and FIG. 9d shows the maximum areal biomass productivity (gm⁻²d⁻¹) of each PBR;

FIGS. 10a to 10d show the results of investigating the shading effect on multiple PBR array. Specifically, FIGS. 10a, 10b, and 10c show the results of investigating the shading effect when sunlight was irradiated at different solar altitude angles of 43° (10 AM), 78° (2 PM), and 22° (6 PM), respectively, and FIG. 10d shows the cell growth of Synechococcus 2973-phaCAB for each PBR; and

FIGS. 11a-11c show the results of operating a 2-ton PBR. Specifically, FIG. 11a is a panoramic photograph of a 2-ton PBR containing Synechococcus 2973-phaCAB, FIG. 11b shows areal biomass yields of Synechococcus 2973-phaCAB in a 2-ton scale PBR, and FIG. 11c shows regional cell growth differences in a 2-ton scale PBR.

### DETAILED DESCRIPTION OF THE INVENTION

Other objects, advantages, and novel features of the invention will become more apparent from the following detailed description and preferred embodiments with reference to the appended drawings. In the drawings, the same elements are denoted by the same reference numerals even though they are depicted in different drawings. Although such terms as “first” and “second,” etc. may be used to describe various elements, these elements should not be limited by above terms. These terms are used only to distinguish one element from another. In the description of the present invention, detailed explanations of related art are omitted when it is deemed that they may unnecessarily obscure the essence of the present invention.

Preferred embodiments of the present invention will now be described in detail with reference to the accompanying drawings.

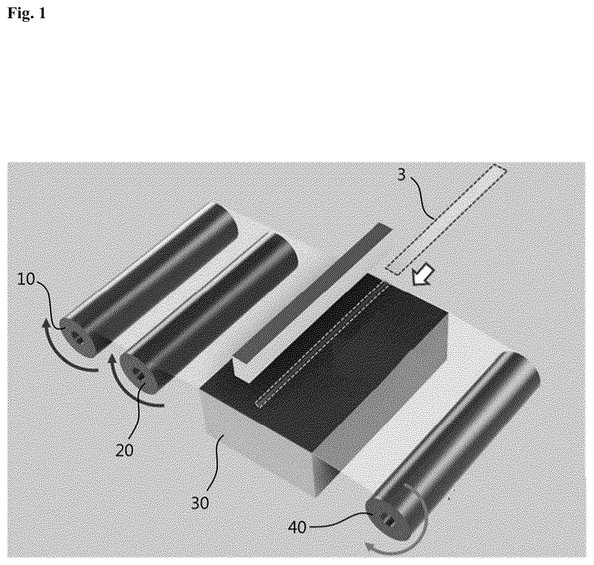

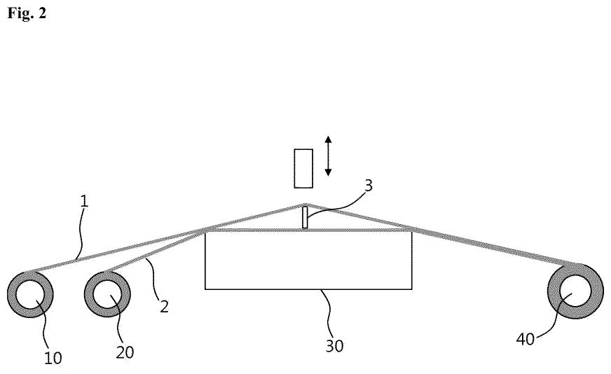

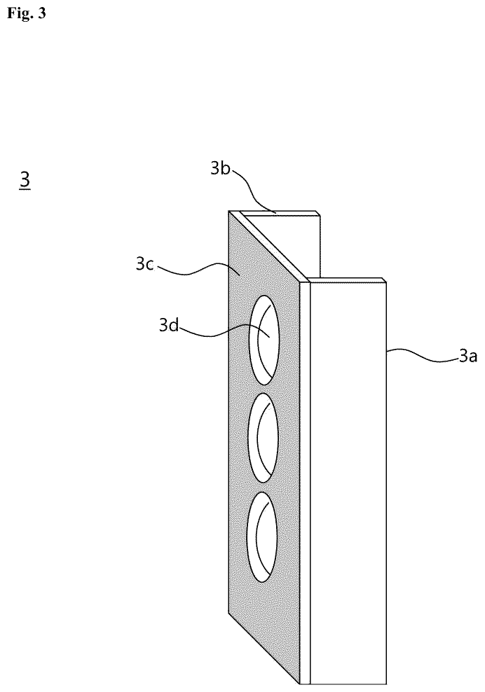

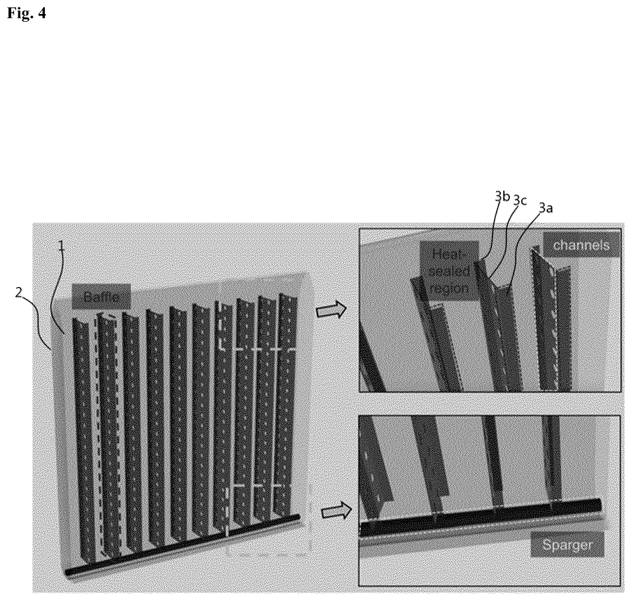

FIG. 1 is a perspective view schematically illustrating a photobioreactor fabrication system according to a first embodiment of the present invention, FIG. 2 is a side view schematically illustrating the photobioreactor fabrication system according to the first embodiment of the present invention, FIG. 3 is a perspective view of the baffle illustrated in FIG. 2, and FIG. 4 illustrates a photobioreactor fabricated using the photobioreactor fabrication system according to the first embodiment of the present invention.

As illustrated in FIGS. 1 and 2, the photobioreactor fabrication system includes a first unwinder 10 continuously unwinding a first transparent film 1, a second unwinder 20 arranged parallel to the first unwinder 10 and continuously unwinding a second transparent film 2 such that the second film 2 faces the first film 1, a heat sealer 30 pressurizing and heat sealing the first 1 and second films 2 to a baffle 3 arranged between the first 1 and second films 2 facing each other, and a rewinder 40 simultaneously and continuously winding the first 1 and second films 2 having passed through the heat sealer 30.

The present invention is directed to a photobioreactor fabrication system in which a hybrid of a roll-to-roll winder and a pressure heat sealer is used to continuously fabricate large-capacity photobioreactors. The photobioreactor fabrication system includes a first unwinder 10, a second unwinder 20, a heat sealer 30, and a rewinder 40, as described above.

The first unwinder 10 continuously unwinds a first film 1 made of a transparent material. The unwound first film 1 is transferred to the heat sealer 30, passes through the heat sealer 30, and is wound on the rewinder 40. The first unwinder 10 is arranged downstream of the transfer direction of the first film 1 along which the photobioreactor fabrication process is carried out. The first unwinder 10 is embodied as a first feed roller that is inserted into a film roll on which the first film 1 is wound, holds the film roll, and unwinds and feeds the first film 1 while rotating. The first feed roller may be connected to a first roller driving means (not illustrated) such as a motor that provides a separate rotational driving force or may be rotated by the rotational driving force of the rewinder 40 that winds the first film 1. The rotational direction of the first feed roller connected to the first roller driving means can be switched such that the first film 1 can be not only unwound from the first unwinder 10 but also wound on the first unwinder 10.

The second unwinder 20 continuously unwinds a second film 2 made of a transparent material. The unwound second film 2 is transferred to the heat sealer 30, passes through the heat sealer 30 to overlap the first film 1, and is wound on the rewinder 40. The second unwinder 20 is arranged parallel to the first unwinder 10 downstream of the transfer direction of the second film 2 along which the photobioreactor fabrication process is carried out. The second unwinder 20 is arranged between the first unwinder 10 and the heat sealer 30 such that the second film 2 underlies the first film 1. The first 1 and second films 2 are transferred to the heat sealer 30 while facing each other. The second unwinder 20 is embodied as a second feed roller that is inserted into a film roll on which the second film 2 is wound, holds the film roll, and unwinds and feeds the second film 2 while rotating. The second feed roller may be connected to a second roller driving means (not illustrated) such as a motor that provides a separate rotational driving force or may be rotated by the rotational driving force of the rewinder 40 that winds the second film 2. The rotational direction of the second feed roller connected to second first roller driving means can be switched such that the second film 2 can be not only unwound from the second unwinder 20 but also wound on the second unwinder 20.

The first 1 and second films 2 are materials for a culture vessel of a photobioreactor. Materials for the first 1 and second films 2 are not particularly limited as long as they are transparent to facilitate the growth of photosynthetic organisms and have high transmittance. For example, the first 1 and second films 2 may be selected from the group consisting of low density polyethylene (LDPE) films, polyethylene terephthalate/cast polypropylene (PET+CPP) films, polyoxymethylene (POM) films, polycarbonate (PC) films, polyester sulfone (PES) films, polyethylene (PE) films, polyvinyl chloride (PVC) films, polyethylene terephthalate (PET) films, polypropylene (PP) films, polyphenylene oxide (PPO=PPE) films, and low density polyethylene/polyethylene terephthalate/Nylon 8 films. These transparent films are lightweight and have high mechanical strength while possessing transmittances comparable to glass and acrylic films that are widely used as materials for culture vessels of photobioreactors. Moreover, the use of the transparent films is advantageous in that a large-capacity culture vessel can be easily produced through pressurization and heat sealing.

The heat sealer 30 is a device that pressurizes and heat seals a baffle 3 to the first 1 and second films 2. The heat sealer 30 is implemented by a heating rack and a heating bar that are heated by an external power source. The heating bar is movable in the vertical direction. The heat sealer can press and heat seal the films arranged between the heating bar and the heating rack while the heating bar moves downward toward the heating rack. However, the heat sealer 30 is not necessarily implemented by a heating rack and a heating bar and is not particularly limited as long as the device can pressurize and heat seal the films.

For heat sealing using the heat sealer 30, the baffle 3 is arranged between the facing first 1 and second films 2 that are unwound from the first 10 and second unwinders 20, respectively, and transferred to the heat sealer 30. Here, the heat sealer 30 pressurizes the regions of the first 1 and second films 2 corresponding to the location of the baffle 3 toward the baffle 3. As a result of the pressurization, one side of the baffle 3 is heat sealed to the first film 1 and the other side of the baffle 3 is heat sealed to the second film 2.

The baffle 3 is a member that is arranged in a culture vessel of a photobioreactor to divide the internal space of the culture vessel (see FIGS. 3 and 4). The baffle 3 may be used to divide the interior of the culture vessel, achieving increased culture capacity. For example, the baffle 3 may include a first heat sealing member 3a, a second heat sealing member 3b, and a partition member 3c. The first heat sealing member 3a is heat sealed to the first film 1, the second heat sealing member 3b is spaced apart from the first heat sealing member 3a and is heat sealed to the second film 2, and the partition member 3c connects the first 3a and second heat sealing members 3b and divides a space between the first 1 and second films 2. The baffle 3 may be formed with a plurality of through holes 3d penetrating the partition member 3c in the thickness direction. The through holes 3d form channels between the spaces formed in the culture vessel divided by the baffle 3. Culture medium components in the culture vessel and gases can move between the divided spaces through the channels. However, the baffle 3 is not necessarily constructed as above and is not limited to a particular construction as long as its both sides can be heat sealed to the first 1 and second films 2 such that the internal space of the culture vessel is divided.

The baffle 3 forms compartments in predetermined inner portions of the front and rear sides of the culture vessel to divide the internal space of the culture vessel. The division of the internal space means that the internal space of the culture vessel is linearly divided in the longitudinal direction from a portion spaced a certain distance from the top of the culture vessel to a portion a certain distance from the bottom of the culture vessel. That is, the internal space of the culture vessel is not divided from the top to the bottom of the culture vessel. As a result of the division, a culture medium can move through the passages formed at regular intervals in the culture vessel. A sparger is arranged at the bottom of the culture vessel to disperse gas into the culture vessel.

The baffle 3 may be provided in plurality. In this case, one of the baffles 3 is inserted and heat sealed, the first 1 and second films 2 are transferred, and the subsequent baffle 3 is inserted and heat sealed. This procedure may be repeated. The distance between the baffle 3 and the subsequent baffle 3 may be appropriately selected depending on desired factors (e.g., capacity) of the reactor.

The rewinder 40 simultaneously and continuously winds the first 1 and second films 2 having passed through the heat sealer 30. The rewinder 40 is arranged upstream of the transfer direction of the first 1 and second films 2 along which the photobioreactor fabrication process is carried out. The rewinder 40 is embodied as a winding roller that winds the first 1 and second films 2 while rotating. The winding roller may be connected to and rotated by a driving means (not illustrated) such as a motor that provides a separate rotational driving force.

Since the rewinder 40 operates in conjunction with the first 10 and second unwinders 20, appropriate tensions can be maintained in the first 1 and second films 2 during transfer.

Since the first 1 and second films 2 are heat sealed only to the baffle 3, the edge portions of the first 1 and second films 2 can be superimposed on and bonded to each other to manufacture a photobioreactor, specifically a culture vessel. The edge portions may also be heat sealed under pressure. Photosynthetic microorganisms may be cultivated in the photobioreactor. Examples of such microorganisms include microalgae, cyanobacteria, and photosynthetic bacteria.

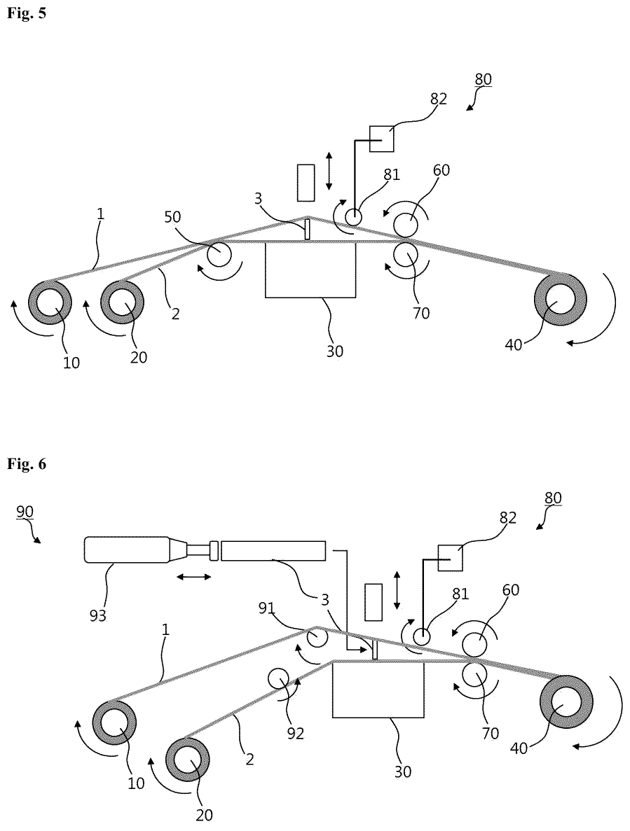

FIG. 5 is a side view schematically illustrating a photobioreactor fabrication system according to a second embodiment of the present invention.

Referring to FIG. 5, the photobioreactor fabrication system may further include a first guide roller 50. The first guide roller 50 rotates while supporting both the first 1 and second films 2 unwound from the first 10 and second unwinders 20, respectively. Thus, the first 1 and second films 2 are superimposed on each other while passing through the first guide roller 50 and are guided to the heat sealer 30. The first guide roller 50 may be rotated by a rotational force received from a separate driving means (not illustrated). Alternatively, the first guide roller 50 in contact with the second film 2 may be rotated by a frictional force generated during transfer of the second film 2.

The photobioreactor fabrication system may further include a second guide roller 60 and a third guide roller 70. The second guide roller 60 and the third guide roller 70 are arranged between the heat sealer 30 and the rewinder 40 to face each other in the vertical direction. The first 1 and second films 2 having passed through the heat sealer 30 are arranged between the second guide roller 60 and the third guide roller 70. The second guide roller 60 and the third guide roller 70 rotate while pressurizing the first 1 and second films 2 therebetween. Thus, the first 1 and second films 2 are superimposed on each other by the second guide roller 60 and the third guide roller 70 and are fed to the rewinder 40. The rewinder 40 easily winds the first 1 and second films 2 simultaneously thereon.

The photobioreactor fabrication system may further include a sensor unit 80. The sensor unit 80 measures the moving distances of the first 1 and second films 2 transferred after being unwound from the first 10 and second unwinders 20, respectively. Based on the measured moving distances, a separate control unit (not illustrated) can control the first 1 and second films 2 to move by preset distances. Here, the preset distances may be the intervals between the baffle 3 and one or more subsequent baffles 3. Thus, repeated processes for baffle insertion and heat sealing can be automated.

The sensor unit 80 may be implemented by a rotator 81 and an operation circuit 82. The rotator 81 is a member that rotates in contact with one of the first 1 and second films 2 moving while facing each other. The rotator 81 is not connected to a separate driving means. The rotator 81 in contact with the first 1 or second film 2 is rotated by a frictional force generated during transfer of the film. The operation circuit 82 measures the rotation angle of the rotator 81 to calculate the moving distances of the first 1 and second films 2. For example, the operation circuit 82 may use electromagnetism induced by the rotating rotator 81 or light transmitted through or reflected by the rotator 81 to measure the rotation angle of the rotator 81. Based on the measured rotation angle, the moving distances of the first 1 and second films 2 can be calculated.

FIG. 6 is a side view schematically illustrating a photobioreactor fabrication system according to a third embodiment of the present invention. As illustrated in FIG. 6, the photobioreactor fabrication system may further include a baffle insertion unit 90.

The baffle inserting unit 90 inserts and arranges the baffle 3 between the first 1 and second films 2 facing each other. The baffle insertion unit 90 may include a first spacer roller 91, a second spacer roller 92, and a baffle feeder 93. The first spacer roller 91 guides the first film 1 to the heat sealer 30 while rotating in contact with the lower surface of the first film 1 unwound from the first unwinder 10. The second spacer roller 92 guides the second film 2 to the heat sealer 30 while rotating in contact with the upper surface of the second film 2 unwound from the second unwinder 20. The second spacer roller 92 spaces the second film 2 from the first film 1. Accordingly, the operation of the first 91 and second spacer rollers 92 forms a space between the first 1 and second films 2. The baffle 3 can be inserted through the space. The baffle feeder 93 feeds the baffle 3 between the first 1 and second films 2 spaced apart from each other. The baffle feeder 93 may be implemented by a cylinder through which a piston pushes the baffle 3 and a conveyor through which the baffle 3 is transported.

The present invention will be explained more specifically with reference to the following experimental examples.

### 1. EXPERIMENTAL METHODS

### 1.1. Photobioreactor (PBR) Fabrication

A customized roll-to-roll winding machine-merged heat-sealer system was developed to automate the fabrication of an easy-to-scale-up PBR. The PBR fabrication system illustrated in FIGS. 1 to 4 was manufactured based on the customized system.

Specifically, a polymer film was placed on each of the two unwinders, and the two films were fixed with a stapler, passed through the heat sealer, and fixed to the rewinder roll with a tape so that the films could be wound on the rewinder. The baffle was inserted between the two films on a region where a heating wire of the heat sealer was located, followed by heat sealing under pressure (120° C., 3-sec heating, and 20-sec cooling) to fabricate a photobioreactor. Thereafter, the photobioreactor was transferred from the rewinder toward the unwinders using the roll-to-roll winder. Another baffle was inserted between the films and heat sealed. This procedure was repeated to fabricate photobioreactors of various scales.

A 70-L PBR consisted of a polymer film (polyethylene terephthalate, polyoctene, and Nylon-6), stone sparger, and gas passage holes. A 150-L and 2000-L easy-to-scale-up PBRs consisted of a polymer film, linear rubber sparger, and baffles. Channels were perforated on the baffle at regular intervals to facilitate culture medium mixing. The linear rubber sparger is located at the bottom of the PBR to supply flue gas containing CO₂.

### 1.2. Cell Culture

1.2.1. Lab Scale Culture of Synechococcus 2973-phaCAB

A mutant of Synechococcus elongatus UTEX 2973-phaCAB (hereafter, Synechococcus 2973-phaCAB) was grown in BG-11 medium (10 mM HEPES-NaOH, pH 8.0) with 50 μgmL⁻¹ of spectinomycin. The Synechococcus 2973-phaCAB seed culture was cultivated under continuous light (200 μmol photons m⁻²s⁻¹) supplied with 5% (v/v) CO₂ in an Erlenmeyer flask with agitation (130 rpm) in an incubator. The temperature was maintained at optimal value (36° C.) for Synechococcus 2973-phaCAB. Optical density (OD) was measured at 730 nm to monitor the cell concentration during cultivation using a UV spectrophotometer (UV-1800, Shimadzu, Japan). Synechococcus 2973-phaCAB cells were inoculated at an initial OD of 0.1.

1.2.2. Outdoor Culture of Synechococcus 2973-phaCAB

A 70-L PBR and easy-to-scale-up PBR (150 L and 2,000 L) were used for outdoor cultivation. The bicarbonate buffer system was formed by 10 M KOH and aeration of flue gas (0.1 vvm; 3-6% CO₂, 11.99±0.73% O₂, 21.72±3.72 ppm NOₓ, 1.43±4.03 ppm CO, water vapor, and dust) in water filled in the PBR. The final pH was 7.8 to 8.0. Smartro NPK (ECOTECH Co.), an N-supply medium, was added to the PBR. In outdoor cultivation, the initial OD730 was 0.02, and the Synechococcus 2973-phaCAB seeds were inoculated into the PBR. Natural sunlight as the sole light source was provided during the daytime in summer, with a light intensity variation of 3.8-1024.1 μmol photons m⁻²s⁻¹ during outdoor cultivation. Moreover, the temperature was maintained at 34-42° C. in a glass greenhouse. Finally, 0.5 mM isopropyl-d-thiogalactopyranoside (IPTG) was added for induction at an OD730 of 0.2.

### 1.2.3. Mutant DNA Analysis

To confirm the inserted PHB gene cassette in Synechococcus 2973-phaCAB, DNA analysis was performed utilizing polymerase chain reaction (PCR). For PCR analysis, cell suspensions from the PBRs were centrifuged (6,000 rpm, 6 min) to obtain cell pellets. Genomic DNA was extracted from the cell pellet by lysing the cells with 5% Chelex-100 chelating resin (Bio-Rad). The NS1 site of Synechococcus 2973-phaCAB was identified using an NS1 primer set. Agarose gel electrophoresis (120 V, 40 min) was performed to confirm the NS1 length of Synechococcus 2973-phaCAB.

### 1.3. Quantification of Biomass and PHB

### 1.3.1. Quantification of Dry Weight

The dry cell weight of the cyanobacterial cells was measured using the gravimetric method. The Whatman, pore size 0.45 μm microfilter paper was rinsed with deionized (DI) water and dried in an oven overnight until the water was completely removed. The initial weight of the rinsed dry paper was measured. After removing the salt remaining on the dry paper with DI water, 3 mL of the cell suspension was loaded. The cell-loaded paper was dried overnight in an oven and the final weight was measured. The dry cell weight was determined as the difference between the initial and final weights of the microfilter paper divided by the cell-loaded volume.

### 2.3.2. Quantification of PHB

Gas chromatography-mass spectrometry (GCMS) (Agilent 6890/5975) equipped with a DB-23 capillary column (60 m×250 μm×0.15 μm) was used for PHB quantification. The following pretreatment was performed to convert PHB to volatile hydroxycarboxylic acid methyl ester, a stable monomer, in a GC column using the acidic methanolysis method. The cell suspension (5 mL) was loaded into a 15 mL glass tube and centrifuged for 10 min at 3000 rpm. After removing the supernatant, the cell pellet was resuspended in 1 mL of chloroform and 1 mL of methanol supplemented with 15% (v/v) H₂SO₄ and incubated at 100° C. for 2 h 30 min. After cooling the glass tube, 1 mL DI water and 1 mL chloroform supplemented with 0.2% (v/v) methyl benzoate were added. The reaction mixture was thoroughly vortexed to separate the organic phase contained in the chloroform layer, and the bottom organic phase was used for PHB analysis. For calibration, methyl benzoate was used as the internal standard, and poly((R)-3-hydroxybutyric acid) (Sigma-Aldrich, USA) was used as the reference standard.

### 1.4. Tracer Test

A tracer test was performed to quantify the mass transfer rate in the PBR by diffusion. A 1 M KOH was used as a tracer. After injecting the tracer, pH shift caused by diffusion of the tracer was measured by collecting the solution from the sampling line installed on each column. Sampling of the solution contained in PBR was performed every 10 min.

### 1.5. Bubble Size Measurement

A diameter of gas bubble formed by aeration was measured by high-speed photography. The shutter speed of the camera (Canon 700D) was set to 1/4000 sec, and an LED was installed behind the PBR to compensate for the insufficient amount of light owing to the fast shutter speed. The average diameter was determined through statistical calculation by comparing the photographed gas bubble diameter with the scale bar.

### 2. RESULTS AND DISCUSSION

### 2.1. Scale Up of PBR Using Roll-to-Roll Winding Machine

A vertical-hanging-type PBR made with polymer film was limited in its application to large-scale process construction owing to the difficulty in automated mass production, even though it is cheaper than a PBR composed of glass or acrylic tubes. A roll-to-roll winding machine-based PBR fabrication system (R2R-PBR fabrication system) was developed to facilitate the mass production of vertical-hanging-type PBR through semi-automation (FIG. 1). Automation of PBR fabrication was realized by introducing a simple and repeatable manufacturing technique using heat-sealing baffles at regular intervals between two polymer films. In addition, because the working volume of the PBR increases in proportion to the number of heat sealing of the baffles, the scale-up of the PBR has been simplified. By introducing a mechanized PBR production system, the manpower required for PBR manufacturing was reduced and the quality of the fabricated PBR was improved. Furthermore, photosynthetic PHB cost can be reduced by decreasing labor costs.

When the PBR manufactured using the R2R-PBR fabrication system was filled with culture medium, the thickness of the PBR was kept uniform by the heat-sealed baffles (FIG. 4). The baffle was perforated with several channels to improve the mass-transfer rate by diffusion within the PBR. CO₂ was effectively delivered into the culture medium using a gas sparger installed at the bottom of the PBR in the longitudinal direction.

### 2.2. Determination of the Number of Channels Perforated on the Baffle to Maximize Mixing Efficiency

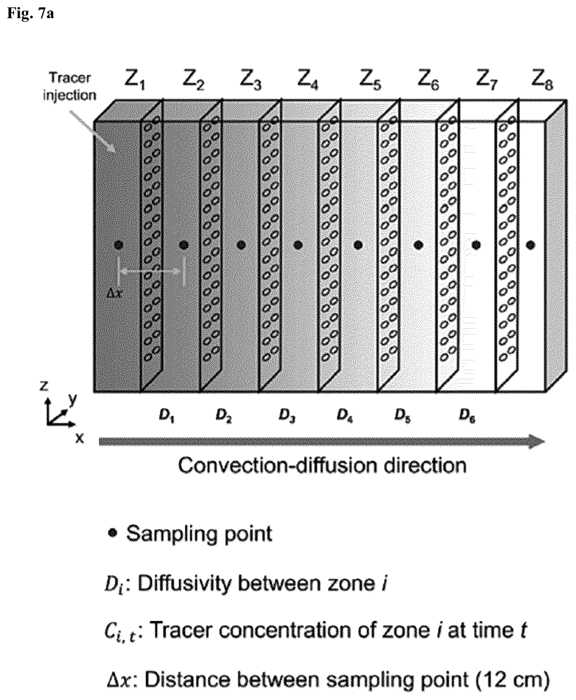

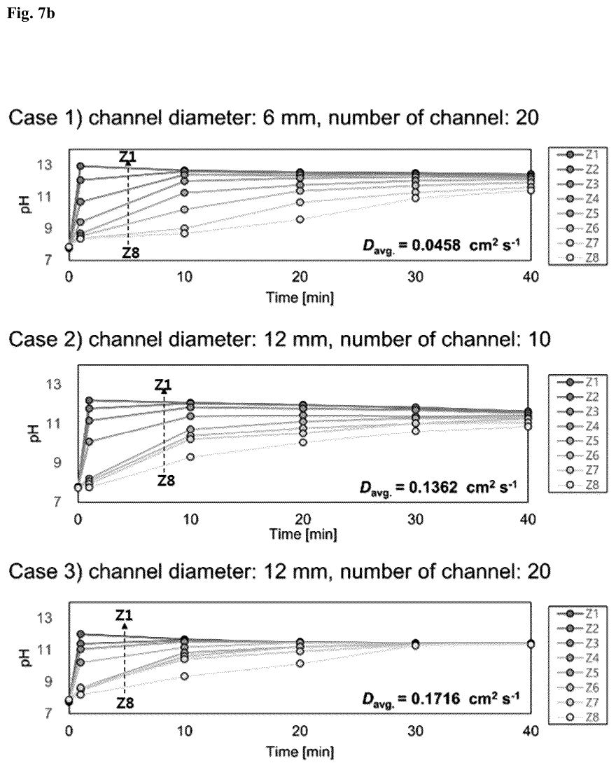

The higher mixing efficiency of the PBR eliminates the gradient of nutrients in the culture medium and guarantees that microalgal cells are uniformly exposed to sunlight. To create an optimal microalgal cell growth environment by maximizing the mixing efficiency in the PBR, the mass-transfer rate was investigated using a tracer test according to the diameter and number of channels formed in the baffle. FIG. 7a is a schematic diagram of the tracer test to quantify the mass transfer rate of PBR and FIG. 7b shows the test results.

Referring to FIG. 7a, the diffusion rate was measured by dividing the 1 m long PBR into eight zones (Z1-Z8) and measuring the concentration of the tracer. The flow of the culture medium in the bubble-column-type PBR was caused by gas bubbles generated from the sparger. Because the flow is induced by the upward movement of the gas bubble, it is difficult for the culture medium to move in the longitudinal direction of the PBR. Because nutrients contained in the PBR are spread by diffusion due to the difference in concentration rather than the flow of the medium, it is crucial to improve the diffusion rate by optimizing the diameter and number of channels formed on the baffle so that diffusion occurs smoothly.

Referring to FIG. 7b, KOH diffused to zone 4 of the PBR within 10 min when 20 channels with a diameter of 6 mm were perforated on the baffle. Although the initial diffusion rate was fast, the difference in pH between zone 1 and zone 8 was 1.04, even at 40 min after tracer injection, indicating that the difference in hydroxide ion concentration was still more than 10 times. When the total channel area was doubled by doubling the diameter (12 mm) of the channel and decreasing the number of channels to 10, the pH change rate of zone 8 was further improved. In this case, it was confirmed that the difference in tracer concentration between zone 1 and zone 8 was approximately 5.8 times at 40 min after the tracer injection. However, when the number of channels with a diameter of 12 mm was increased to 20, the pH in all zones reached equilibrium at 30 min after tracer injection.

Furthermore, the average diffusivity (D_{avg}) for the channels formed in the baffle was calculated using the convection-diffusion transport equation:

∂C/∂t=D(∂²C/∂x²), (1)

where C denotes the concentration of hydroxide ions ([OH⁻]) used as a tracer, and the concentration of hydroxide ions was calculated from the pH. The convective-diffusion transport equation was converted into the following difference equation and used for numerical calculations of D_{avg}:

$${D}_{i} = ( \frac{{C}_{i - 1 , t + 1} - 2 {C}_{i , t + 1} + {C}_{i + 1 , t + 1}}{Δ {x}^{2}} ) / ( \frac{{C}_{i , t + 1} - {C}_{i , t}}{Δ t} ) , \qquad ( 2 )$$

where C_{i,t} is the concentration of hydroxide ions in zone i at time t.

The D_{avg} values for each case were 0.0458, 0.1362, and 0.1716 cm²s⁻¹, respectively, which proves that each PBR has mass-transfer characteristics consistent with the results of the tracer test. The D_{avg} of case 2 was 80% of that of case 3, but the actual diffusion rate of case 2 was significantly slower than that of case 3. In case 2, the diffusion rate from zone 1 to zone 4 was remarkably improved compared with that in case 1, but the tracer diffusion rate from zone 6 to zone 8 was still low. Therefore, the overall equilibrium of the tracer concentration was reached late, even though D_{avg} of case 2 was 80% of that of case 3.

### 2.3. Gas-Liquid Mass Transfer Characteristics

The electricity required to blow flue gas containing CO₂ into the reactor was the major driving energy of the bubble column-type PBR. The efficient use of the energy required for flue gas aeration could considerably affect the economic feasibility of the cyanobacteria derived PHB production process. The bubble diameter (d_{b}), gas hold-up (ε_{G}), and interfacial area (a) are important parameters that affect the growth of photosynthetic microorganisms in PBR. The gas-liquid mass transfer parameters and the corresponding flow rate are summarized in Table 1.

TABLE 1

| Flow rate | d_{b, mean} | | a | P_{G}/Vₗ |
|---|---|---|---|---|
| [vvm] | [mm] | ε_{G} | [m³ m⁻²] | [W m⁻³] |
| 0.05 | 1.87 ± 0.43 | 0.0188 | 55.14 | 19.29 |
| 0.10 | 2.00 ± 0.48 | 0.0309 | 85.42 | 39.02 |
| 0.20 | 2.31 ± 0.55 | 0.0368 | 89.29 | 58.86 |

### 2.3.1. Bubble Diameter (d_{b})

In the culture medium, CO₂ gas transfer occurs on the surface of the gas bubble, which corresponds to the gas-liquid interface. Therefore, CO₂ transfer can occur effectively as the d_{b} decreases. However, d_{b} tended to increase in proportion to the volumetric flow rate (vvm). When the flow rate was increased 4-fold, d_{b} grew up by 23%, resulting in a 20% decrease in the surface area per unit volume of the gas bubble. An increase in d_{b} in accordance with the flow rate was previously observed in a porous metal sparger.

### 2.3.2. Gas Hold-Up (ε_{G})

Gas hold-up is a factor that indicates the volume fraction occupied by the gas phase in the liquid-gas reactor and is one of the most important hydrodynamic characteristics of the PBR. The gas hold-up indicates the volume ratio occupied by the gas phase; hence, it indicates the residence time of the flue gas in the PBR. Because the gas hold-up is proportional to the superficial velocity, an increase in the gas flow rate causes an increase in the gas hold-up. It has been confirmed that the PBR developed in the present invention had twice the gas hold-up compared with the previously reported vertical hanging-bag-type PBR even under low flow rate conditions. Because a cylinder-shaped stone sparger (D=10 mm, H=20 mm) installed in the previously reported PBR had insufficient pores to form bubbles, the gas hold-up was low, even under a higher gas flow rate (0.3 vvm). However, the gas hold-up was effectively improved by installing a linear porous sparger (D=30 mm, H=1000 mm) with sufficient pores in the longitudinal direction of the PBR.

### 2.3.3. Interfacial Area (a)

The interfacial area represents the total area of contact between the liquid and the gas in a liquid-gas operation. An increase in the interfacial area is advantageous for CO₂ delivery to cyanobacteria because the contact area between the culture medium and the aerated flue gas is proportional. The interfacial area can be estimated from the Sauter mean diameter (d_{bs}, Eq. (3)), and gas hold-up (Eq. (4)).

d_{bs}=Σnᵢd_{b}³/Σnᵢd_{b}² (3)

a=6ε_{g}/d_{bs}(1−ε_{g}) (4)

When the gas flow rate was increased from 0.05 vvm to 0.10 vvm, the interfacial area was improved by 55%. In contrast, when the gas flow rate doubled from 0.10 vvm, the increase in the interfacial area was only approximately 5%. It has been previously reported that the gas hold-up improvement efficiency decreases as the gas flow rate increases. This phenomenon can be explained by an analogy with Ohm's law. In the aeration process using a porous sparger, a pressure drop (resistance) occurs when flue gas passes through the pores of the sparger. A higher pressurization (voltage) is required to efficiently transport the flue gas with an increased flow rate (current) through the pores. However, the pressure of the flue gas transported by a ring blower was insufficient to effectively pass through the pores with a higher flow rate (0.20 vvm).

### 2.3.4. Energy Efficiency

By comparing the energy required for flue gas aeration (Eq. (5)) and the interfacial area, the optimal flow rate for energy-effective PBR operation can be determined:

P_{G}/Q_{L}=ρ_{L}gU_{G}, (5)

where ρ_{L}, g, and U_{G} represent the water density, gravitational acceleration, and superficial gas velocity, respectively. The power consumption increases in proportion to the gas flow rate. However, the energy consumed for aeration was not efficiently reflected in improving the interfacial area at a gas flow rate of 0.20 vvm. Therefore, a gas flow rate of 0.10 vvm was determined to be the most efficient operation mode considering the interfacial area and power consumption.

### 2.3.5. Comparison of CO₂ Transfer Efficiencies Between PBRs

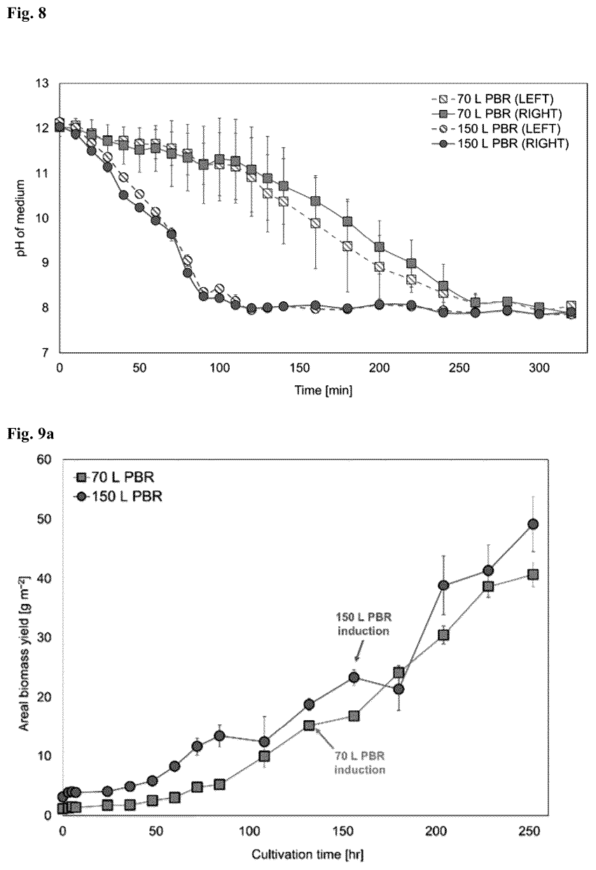

The CO₂ transfer efficiencies of the control hanging-bag-type PBR (serial column PBR, 70 L PBR) and the newly developed PBR (150 L PBR) were compared. As the 10 mM KOH solution in the PBR reacts with the CO₂ contained in the flue gas, carbonate ions are formed, and the pH of the solution decreases. The faster the CO₂ transfer rate from the flue gas into the solution, the sooner the pH of the solution reaches equilibrium. After blowing the flue gas into the 70-L PBR and 150-L PBR, the pH of the medium in both end columns was monitored. The pH tracking experiment was conducted in duplicate. The results are shown in FIG. 8. FIG. 8 compares CO₂ transfer efficiencies between the 70-L PBR (control) and the 150-L PBR (newly developed).

It was confirmed that the rate of pH change in the 150-L PBR was more rapid than that in the 70-L PBR. Approximately 100 min after blowing flue gas into the 150-L PBR, the pH of the solution reached 8, which is the optimum pH for Synechococcus 2973-phaCAB. However, in the 70-L PBR, the pH reached the target equilibrium value of 8 after 250 min. Even though the flue gas was injected at the same gas flow rate (0.10 vvm), the 150-L PBR reached equilibrium 2.5 times faster than the 70-L PBR. It could be analyzed that the CO₂ transfer efficiency in the 150-L PBR was enhanced in proportion to the rate of reaching the pH equilibrium when compared with the existing reactor.

### 2.4. Comparison of PHB Producing Performance Between PBRs

### 2.4.1. Biomass Productivity

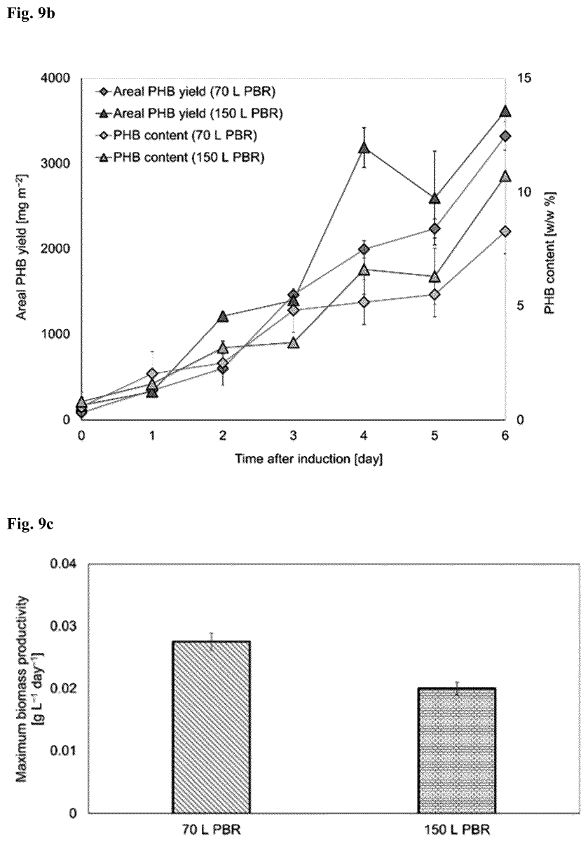

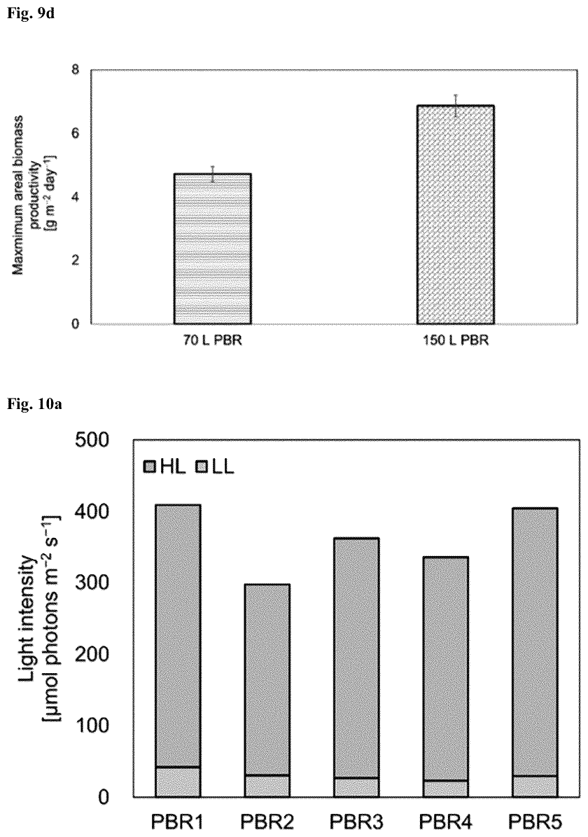

The biomass productivity of the 70-L PBR and 150-L PBR was compared under the same sunlight conditions. The 70-L PBR consisted of six columns with a diameter of 12 cm, while the 150-L PBR had a panel shape with a thickness of 14 cm. FIGS. 9a to 9d compare areal biomass productivities and PHB productivities between the 70-L PBR (control) and the 150-L PBR (newly developed). Specifically, FIG. 9a shows the biomass production per area of each PBR, FIG. 9b shows the PHB production per area and PHB content (%) of each PBR, FIG. 9c shows the maximum biomass productivity (gL⁻¹d⁻¹) of each PBR, and FIG. 9d shows the maximum areal biomass productivity (gm⁻²d⁻¹) of each PBR.

The intensity of the sunlight passing through the culture medium in the PBR was attenuated exponentially according to the Beer-Lambert law. Therefore, photosynthesis occurs more actively in cyanobacteria contained in the 70-L PBR than in the 150-L PBR. The maximum cell growth rate of the 70-L PBR was 37% higher than that of the 150-L PBR because of the difference in light utilization (FIG. 9c). Because the culture medium volume per unit area of the 150-L PBR was twice that of the 70-L PBR, the maximum areal productivity of the 150-L PBR was 45% higher (FIG. 9d). During outdoor cultivation, the biomass production per unit area of the 150-L PBR was higher than that of the 70-L PBR. Consequently, the 150-L PBR produced 21% more cyanobacteria biomass containing PHB than the 70-L PBR when cultivation was completed (FIG. 9a). Because areal productivity is a major parameter of life cycle assessment to evaluate the cost of biomass production, improving the areal productivity of cyanobacteria cultivation systems is essential for the realization of economical feasibility.

### 2.4.2. Cyanobacteria Derived PHB Productivity

When OD730 of Synechococcus 2973-phaCAB cultured in the PBR reached 0.2, the phaCAB gene was overexpressed using 0.5 mM IPTG so that the CO₂ could be converted into PHB through photosynthesis. The PHB content of Synechococcus 2973-phaCAB cultured in 150-L PBR was 10.7 (w/w), which was 29% higher than that in the 70-L PBR (FIG. 9b). IPTG was introduced into the inlet of the PBR and spread into the entire medium through diffusion. Because the nutrient diffusion rate optimization was conducted during the design of the 150-L PBR, IPTG introduced into the PBR was uniformly spread within a culture medium within a short period. As the IPTG concentration of the whole culture medium reached the optimal point for phaCAB gene overexpression, the process manipulation for PHB production was carried out accurately. Therefore, Synechococcus 2973-phaCAB cells cultivated in the 150-L PBR synthesized PHB in an environment optimized for phaCAB gene overexpression. As a result, the 150-L PBR had a higher PHB production performance compared with the 70-L PBR, even though the working volume was doubled. Compared with the previous experimental results in a single column (3 L single column PBR, final PHB content: 10.5%), there was no decrease in PHB production capacity even after a 50-fold scale up. In addition, the maximum areal PHB productivity of the 150-L PBR was 1.087 gm⁻²d⁻¹, which was confirmed to have high performance compared with the previously reported areal PHB productivity.

### 2.5. Shading Effect Due to Multiple PBR Operation

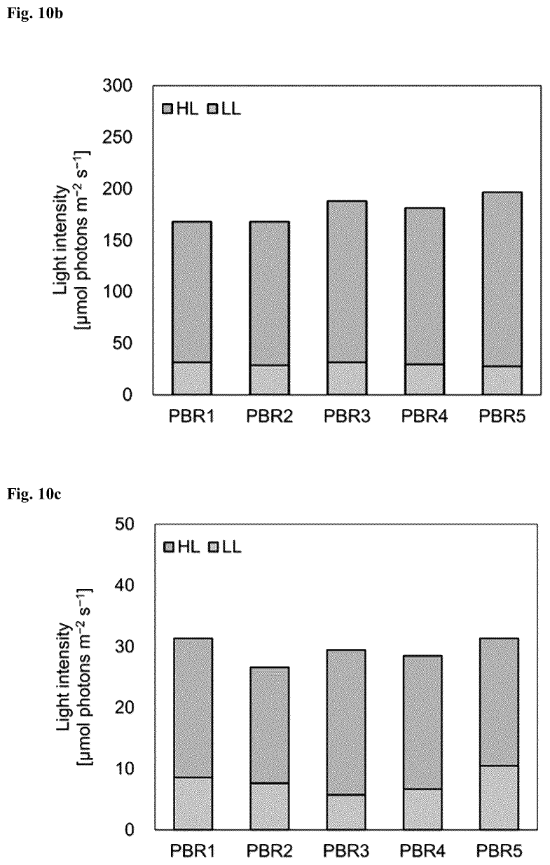

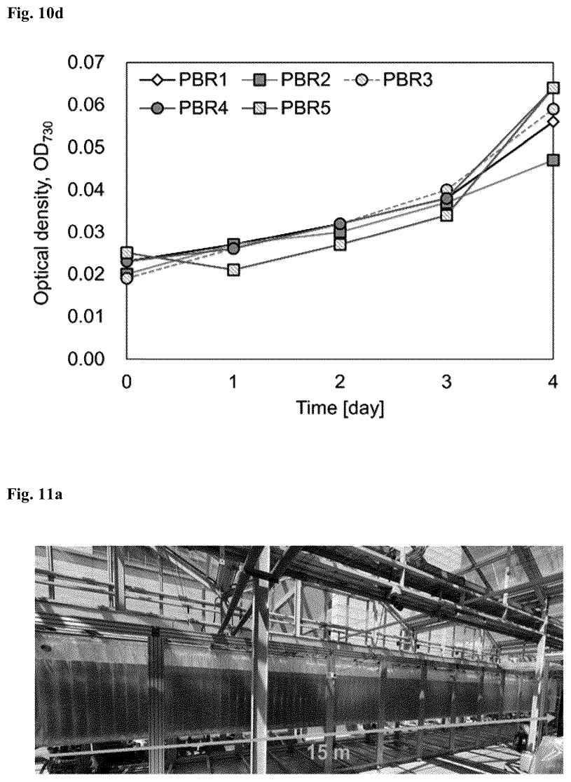

To realize an economically feasible photosynthetic PHB production process, it is necessary to reduce the site area required for cyanobacteria culture by operating multiple PBRs in the form of an array. When multiple PBRs are operated in the form of an array, the photosynthetic efficiency of the PBR can be decreased owing to the shading effect. The intensity of light irradiation of each PBR and growth of Synechococcus 2973-phaCAB were investigated during multiple PBR array operations. The variance in the light intensity due to the solar altitude angle was analyzed by varying the measurement time. The results are shown in FIGS. 10a to 10d. Specifically, FIGS. 10a, 10b, and 10c show the results of investigating the shading effect when sunlight was irradiated at different solar altitude angles of 43° (10 AM), 78° (2 PM), and 22° (6 PM), respectively, and FIG. 10d shows the cell growth of Synechococcus 2973-phaCAB for each PBR.

Before the solar altitude angle reached the meridian altitude, PBR1 received the highest light intensity. PBR2-4, located between the other PBRs, showed a 27% reduction in the intensity of light by shading (FIG. 10a). When the sun was located at the meridian altitude (78°), there was no shadow between the PBRs because the PBR array direction and sunlight irradiation became almost parallel. As the sun moved westward, the intensity of light irradiated on PBR5 was the strongest, and PBR2 received light with a 15% decrease in intensity compared with PBR5. It was confirmed that when the photobiological process was operated in the form of multiple PBR arrays, the outermost PBR1 and PBR5 received consistently high-intensity light. PBR3 received strong light when the sun was located at the meridian altitude. Among the multiple PBR arrays, PBR2 consistently received light with a weaker intensity than the other PBRs.

The difference in light intensity irradiated to the PBR affected the growth rate of Synechococcus 2973-phaCAB, and hence the cell growth rate in PBR2 was lower than that in other PBRs (FIG. 10d). Synechococcus 2973-phaCAB cultured in the PBRs, except for PBR2, showed little growth inhibition due to a lack of light. The biomass production performance of PBR2 was 78% of that of the other PBRs. To improve the productivity of the photosynthetic PHB production process using a hanging-bag-type PBR array, additional research on the process design such as a study of the installation direction of the PBR array optimized for seasonal changes in the altitude of the sun is needed.

### 2.6. Performance of the 2-Ton PBR

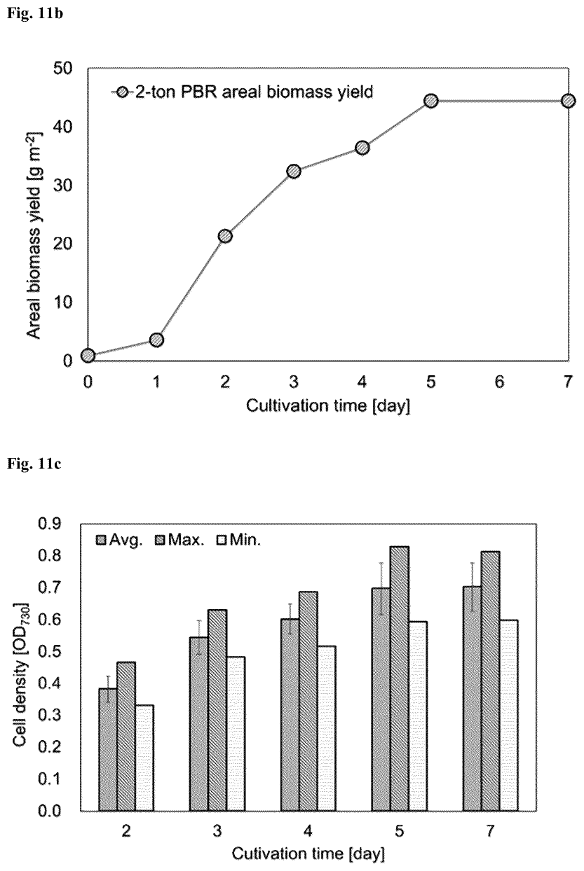

FIGS. 11a and 11b show the results of operating a 2-ton PBR. Specifically, FIG. 11a is a panoramic photograph of a 2-ton PBR containing Synechococcus 2973-phaCAB, FIG. 11b shows areal biomass yields of Synechococcus 2973-phaCAB in a 2-ton scale PBR, and FIG. 11c shows regional cell growth differences in a 2-ton scale PBR.

The module 150-L PBR was scaled up to a 2-ton PBR, and Synechococcus 2973-phaCAB was cultivated. The scale up of the PBR developed in the present invention was achieved by extending the length of the PBR instead of the diameter of the column. Therefore, it was possible to scale up the PBR while maintaining the performance of the module PBR. By comparing the biomass productivity of the 2-ton PBR with that of the 150-L PBR, it was confirmed that there was no deterioration in the cell growth rate due to the scale up (FIG. 11b). The areal biomass yield of 2-ton PBR was 44.44 gm⁻², which was 90% of that of 150-L PBR (FIG. 9a). Considering the cultivation period in 2-ton PBR was 3 days shorter than in 150-L PBR, it could be concluded that there was a little cultivation performance drop due to scale-up. The maximum areal productivity of 2-ton PBR was 17.77 gm⁻²d⁻¹, which was 2.16-fold higher than that of 150-L PBR. Higher biomass productivity was achieved because the amount of sunlight irradiated on the 2-ton PBR was better. The flow of the culture medium by flue gas aeration was the driving force that mixes Synechococcus 2973-phaCAB cultured in the 2-ton PBR. Because the flow of the culture medium by aeration mainly occurred in the direction parallel to gravity, vigorous mixing was difficult to take place in the longitudinal direction of the 2-ton PBR. Therefore, regional differences may occur in the growth of Synechococcus 2973-phaCAB cultivated in the 2-ton PBR owing to uneven irradiation of sunlight by greenhouse structures and clouds. To confirm this effect quantitatively, the difference in cell growth rate in the 2-ton PBR was analyzed by sampling the cell culture from seven zones of the 2-ton PBR. There was no consistent relation between cell density and sampling location. The maximum and minimum values of cell density of cultures collected from 7 zones approximately 15% deviated from the average value (FIG. 11c).

Using a single large-scale PBR, it is possible to reduce the use of subsidiary materials required for PBR production, such as pipes and valves, which has the advantage of reducing process construction cost. Furthermore, the process operation becomes easier because the number of PBRs to be managed is reduced. The easy-to-scale-up PBR developed in the present invention uses a simple manufacturing mechanism and inexpensive raw materials instead of glass and acrylic vessels. Therefore, if a photosynthetic PHB production process is established using this PBR production system, the cost of process construction will be significantly reduced. By reducing the cost of photosynthetic PHB production, it is possible to contribute to the commercialization of bioconversion processes that can simultaneously produce eco-friendly biodegradable plastics and reduce greenhouse gas emissions.

### 3. CONCLUSIONS

In the present invention, a semi-automated, easy-to-scale-up PBR fabrication system was developed. The developed PBR produced 21% more PHB-containing biomass than the existing serial-column PBR in the same area. The PHB content was 29% higher than that of the existing PBR because IPTG-driven PHB production occurred effectively due to mass transfer efficiency enhancement. Synechococcus 2973-phaCAB was successfully cultured in a 2-ton PBR without performance drop due to scale up, and areal biomass productivity reached 17.77 gm⁻²d⁻¹. Consequently, easy-to-scale-up PBR, which has higher areal biomass and PHB productivity, could be applied to improved photosynthetic PHB production process utilizing CO₂.

Although the present invention has been described herein with reference to the specific embodiments, these embodiments do not serve to limit the invention and are set forth for illustrative purposes. It will be apparent to those skilled in the art that modifications and improvements can be made without departing from the spirit and scope of the invention.

Such simple modifications and improvements of the present invention belong to the scope of the present invention, and the specific scope of the present invention will be clearly defined by the appended claims.

What is claimed is:

1. A photobioreactor fabrication system comprising a first unwinder continuously unwinding a first transparent film, a second unwinder arranged parallel to the first unwinder and continuously unwinding a second transparent film such that the second film faces the first film, a heat sealer pressurizing and heat sealing the first and second films to a baffle arranged between the first and second films facing each other, and a rewinder simultaneously and continuously winding the first and second films having passed through the heat sealer.

2. The photobioreactor fabrication system according to claim 1, further comprising a first guide roller superimposing the unwound first and second films on each other and guiding the superimposed first and second films to the heat sealer.

3. The photobioreactor fabrication system according to claim 1, wherein the baffle comprises a first heat sealing member heat sealable to the first film, a second heat sealing member spaced apart from the first heat sealing member and heat sealable to the second film, and a partition member connecting the first and second heat sealing members and dividing a space between the first and second films.

4. The photobioreactor fabrication system according to claim 3, wherein the baffle is formed with a plurality of through holes penetrating the partition member.

5. The photobioreactor fabrication system according to claim 1, further comprising a second guide roller arranged between the heat sealer and the rewinder and a third guide roller arranged to face the second guide roller in the vertical direction through the first and second films having passed through the heat sealer such that the first and second films are superimposed on each other and are guided to the rewinder.

6. The photobioreactor fabrication system according to claim 1, further comprising a sensor unit measuring the moving distances of the first and second films facing each other after being unwound.

7. The photobioreactor fabrication system according to claim 5, wherein the sensor unit comprises a rotator rotating in contact with one of the first and second films moving while facing each other and an operation circuit measuring the rotation angle of the rotator to calculate the moving distances of the first and second films.

8. The photobioreactor fabrication system according to claim 1, further comprising a baffle insertion unit inserting and arranging the baffle between the first and second films facing each other.

9. The photobioreactor fabrication system according to claim 8, wherein the baffle insertion unit comprises a first spacer roller guiding the first film to the heat sealer while rotating in contact with the lower surface of the first film unwound from the first unwinder, a second spacer roller spacing the second film from the first film and guiding the second film to the heat sealer while rotating in contact with the upper surface of the second film unwound from the second unwinder, and a baffle feeder feeding the baffle between the first and second films spaced apart from each other.

10. The photobioreactor fabrication system according to claim 1, wherein the first and second films are selected from the group consisting of low density polyethylene (LDPE) films, polyethylene terephthalate/cast polypropylene (PET+CPP) films, polyoxymethylene (POM) films, polycarbonate (PC) films, polyester sulfone (PES) films, polyethylene (PE) films, polyvinyl chloride (PVC) films, polyethylene terephthalate (PET) films, polypropylene (PP) films, polyphenylene oxide (PPO=PPE) films, and low density polyethylene/polyethylene terephthalate/Nylon 8 films.
